(19) United States
(12) Patent Application Publication
(10) Pub. No.: US 2021/0387691 A1
(43) Pub. Date: Dec. 16, 2021

(54) ELECTRIC VEHICLE WITH CONTACT SURFACE SANITIZING DEVICE

(71) Applicant: Acton, Inc., Livermore, CA (US)

(72) Inventor: Peter Treadway, Livermore, CA (US)

(21) Appl. No.: 17/342,480

(22) Filed: Jun. 8, 2021

Related U.S. Application Data
(60) Provisional application No. 63/038,222, filed on Jun. 12, 2020.

(51) Int. Cl. B62J 50/16; B62K 21/12; B62J 43/10; B62J 43/20

(52) U.S. Cl. CPC B62J 50/16; B62K 21/12; B62K 2202/00; B62J 43/20; B62J 43/10

(57) ABSTRACT

An electric vehicle is provided with a steering element that can include a handle bar element. A platform is configured to support a rider of the electric vehicle. At least one front wheel and at least one rear wheel are included. An electric motor is configured to provide a mechanical power to at least one of the front and rear wheels. A battery housing, is configured to removably receive and hold one or more battery packs. The one or more battery packs are configured to power the electric vehicle. An applicator device is included for dispensing sanitizing layer/coating to at least a portion of the steering element.

### CROSS-REFERENCE TO RELATED APPLICATIONS

This application claims the benefit of U.S. Provisional Patent Application 63/038,222, filed on Jun. 12, 2020.

### BACKGROUND

### Field of the Invention

This invention relates generally to, and more particularly to electric vehicles with a contact surface sanitizing device.

### Description of the Related Art

Numerous proposals have presented for providing wireless communications between vehicles and between a vehicle and roadside units. Examples of such proposals include the IEEE 1609 set of standards for wireless access in vehicle environments (WAVE) and the ETSI standards for intelligent transport systems. In these systems, the communications may help to enhance the safety of the vehicles, provide additional information and provide additional services. For example, the communications may help to avoid collisions, provide collision warnings, enhance traffic flow, provide navigation and route guidance, provide point of interest information and provide remote diagnostics. Numerous other uses of these communications have been proposed as well.

In one method credentials are stored in credential files in a storage in a vehicle. Each credential specifies a private signing key and a digital certificate. The digital certificate is valid for a specified time frame. The credential files are named to contain information regarding the specified time frame in which the credentials in the credential files are valid. A sorted index to the credential files is stored in the storage of the vehicle. The index indexes the credential files by the time frames. The index is used to retrieve the credential files for a duration wherein the credentials in the retrieved credential files are valid. One or more of the retrieved credential files may then be used in the vehicle communication system during the duration.

In another method a local certificate manager is started and upon starting the local certificate manager, a check is made whether enough private keys for a specified period have been rederived. If enough private keys have been rederived, at least some of the rederived private keys are stored in a nonvolatile storage in a vehicle. If not, enough private keys have been rederived, additional private keys are rederived and store in the non-volatile memory in the vehicle. At least one of the private keys is used in securing a communication in the secure vehicle communication system during the specified time period.

In accordance with a further exemplary embodiment, a user request to change identity is received in a secure vehicle communication system. In response to the user request, an indication is provided to the user that certificates are available for selection to realize the identity change. A selection is received from the user. A certificate identifier for the selected one of the certificates is returned to the user and a selected one of the certificates is used for the communication in a secure vehicle communication system.

There is a need for improved vehicles. There is a further need for vehicles, with surfaces of the vehicle that an operator or rider is in contact with, made from antibacterial/antimicrobial plastic, or an associated exterior surface, where the operator or rider contacts, including but not limited to grips, brake levers, and the like, can be treated with an antibacterial/antimicrobial material.

### SUMMARY

An object of the present invention is to provide improved vehicles.

A further object of the present invention is to provide electric vehicles with antibacterial/antimicrobial plastic, or an associated exterior surface, where the operator or rider contacts, including but not limited to grips, brake levers, and the like, can be treated with an an material.

These and other objects of the present invention are achieved in an electric vehicle with a steering element that can include a handle bar element. A platform is configured to support a rider of the electric vehicle. At least one front wheel and at least one rear wheel are included. An electric motor is configured to provide a mechanical power to at least one of the front and rear wheels. A battery housing is configured to removably receive and hold one or more battery packs. The one or more battery packs are configured to power the electric vehicle. An applicator device is included for dispensing sanitizing layer/coating to at least a portion of the steering element.

### BRIEF DESCRIPTION OF THE DRAWINGS

FIG. 1 illustrates an electric scooter according to embodiments of the disclosed technology.

FIG. 2 illustrates further detail of the electric scooter of FIG. 1.

FIG. 3 illustrates further detail of the deck assembly and latch of FIGS. 1 and 2.

FIG. 4 illustrates detail of the scooter of FIGS. 1 and 2 with the latch in an open position.

FIG. 5 illustrates further detail of the scooter of FIGS. 1 and 2 with the latch in an open position.

FIG. 6 illustrates detail of the scooter of FIGS. 1 and 2 with the latch in an open position.

FIG. 7 illustrates further detail of the scooter of FIGS. 1 and 2 during installation, or removal, of the deck assembly.

FIG. 8 illustrates a quick twist electrical soft connector according to embodiments of the disclosed technology.

FIG. 9 illustrates a cushioned electrical connector according to embodiments of the disclosed technology.

FIG. 10 illustrates a compound locking assembly according to embodiments of the disclosed technology.

FIG. 11A illustrates a portion of the scooter during removal or installation of the deck assembly.

FIG. 11B illustrates the retention device for the loose portions of the electrical cables of the scooter.

FIG. 12 illustrates a hidden latch mechanism for the scooter.

FIG. 13 illustrates a process for a user to install a removable deck assembly into an electric scooter according to embodiments of the disclosed technology.

FIG. 14 illustrates a process for a user to remove a removable deck assembly from an electric scooter according to embodiments of the disclosed technology.

FIG. 15 illustrates one embodiment of electric vehicle with public and private keys, and vehicle to vehicle communication.

FIGS. 16-24 illustrates various embodiments of the present invention with UVC light sources.

FIG. 25(a) is an isometric front side view depicting the housing for the embodiment of FIG. 1.

FIG. 25(b) is a front isometric view of an application and dispensing tube insert which fits into the main housing.

FIG. 25(c) is an isometric view of a replacement cart handle cover and rail mechanism used to attach the main housing to the shopping cart handle.

FIG. 26 is a front side view of an alternative preferred embodiment of the invention.

FIG. 27 is a top plan view of the apparatus of FIG. 26.

FIG. 28 is an end view of the apparatus of FIG. 26.

FIG. 29 is an isometric view of the apparatus of FIG. 26 as viewed from the front side.

FIG. 30 is an isometric view of the apparatus of FIG. 26 as viewed from the backside thereof.

FIG. 31 is an isometric backside view depicting the apparatus generally depicted in FIGS. 26-30 as attached to a typical shopping cart handle.

FIG. 32 is an isometric front side view of the apparatus of the invention as attached to a shopping cart handle.

FIG. 33 is an isometric view illustrating the manner of operation of the apparatus of FIGS. 26-32.

FIG. 34 is an isometric view of the apparatus of FIGS. 26-33 depicting the internal construction of the housing reservoir pump and applicators.

FIG. 35 is an exploded isometric view of a further alternative embodiment of the invention.

FIG. 35(a) is an isometric view of the assembled embodiment of FIG. 35.

FIG. 36 is an exploded isometric view of a further embodiment of the invention.

FIG. 36(a) is an isometric view of the assembled embodiment of FIG. 36.

FIG. 37 is a cross sectional view of an alternative construction of such a dispenser.

FIG. 38 illustrates the dispenser of FIG. 37 in a cross-sectional view when actuated, and

FIG. 39 is a partial isometric view of the sanitizing layer/coating dispensing arrangement associated with the valve mechanisms of FIGS. 37 and 38.

### DETAILED DESCRIPTION

Embodiments of the described technology provide electric scooters having top-swappable batteries. The batteries may be attached to the underside of the deck of the scooter to form a removable deck assembly. The deck assembly may be removed from the top of the scooter by operating a latch and lifting a handle of the assembly. The deck assembly may be returned to the scooter in a similar manner.

In some embodiments, the battery may be electrically coupled to a motor of the scooter by electrical cables and an electrical connector. The electrical connector may be a quick twist connector that is opened and closed by twisting its halves in opposite directions.

In some embodiments, instead of using electrical cables, the scooter and deck assembly may include electrical connectors that mate when the deck assembly is installed in the scooter. The electrical connectors may be surrounded by cushions that protect the connectors from micro vibrations, dirt and water, and the like.

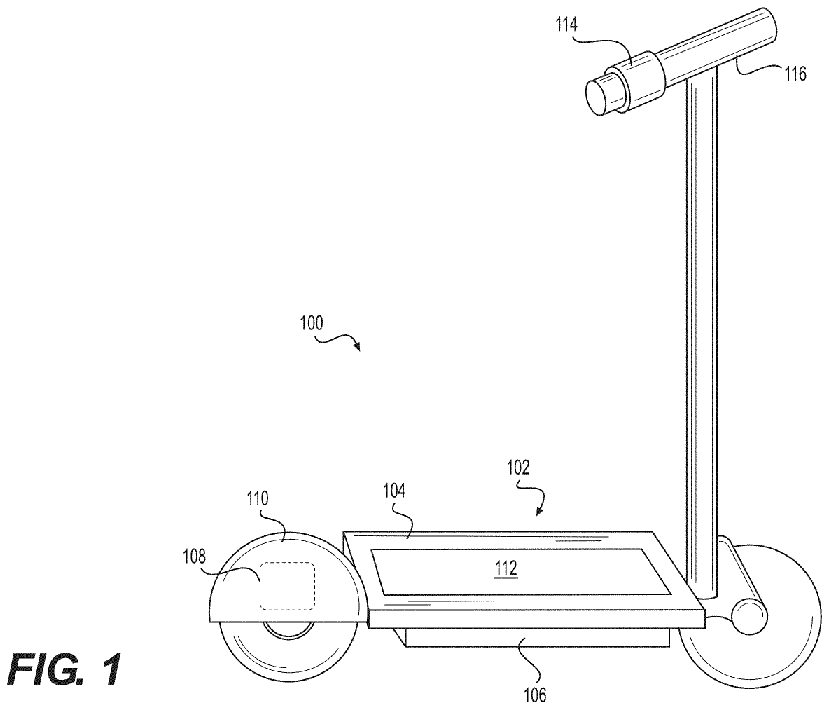

FIG. 1 illustrates an electric scooter 100 according to embodiments of the disclosed technology. Referring to FIG. 1, the scooter 100 includes a deck assembly 102 removably attached to a frame 104 of the scooter 100. The deck assembly 102 includes a battery case 106 mounted underneath a deck 112. The battery case 106 includes one or more batteries (not shown). The batteries are electrically coupled to an electric drive motor 108, which is protected by a housing 110. The scooter 100 may be steered by turning a handlebar 116. The speed of the motor 108 may be controlled using a throttle 114 mounted on the handlebar 116.

The electric scooter 100 is depicted in FIG. 1 as having only two wheels. However, it will be appreciated that the disclosed technology applies to scooters having any number of wheels. Furthermore, it will be appreciated that the disclosed technology applies to vehicles other than scooters, and having any number of wheels.

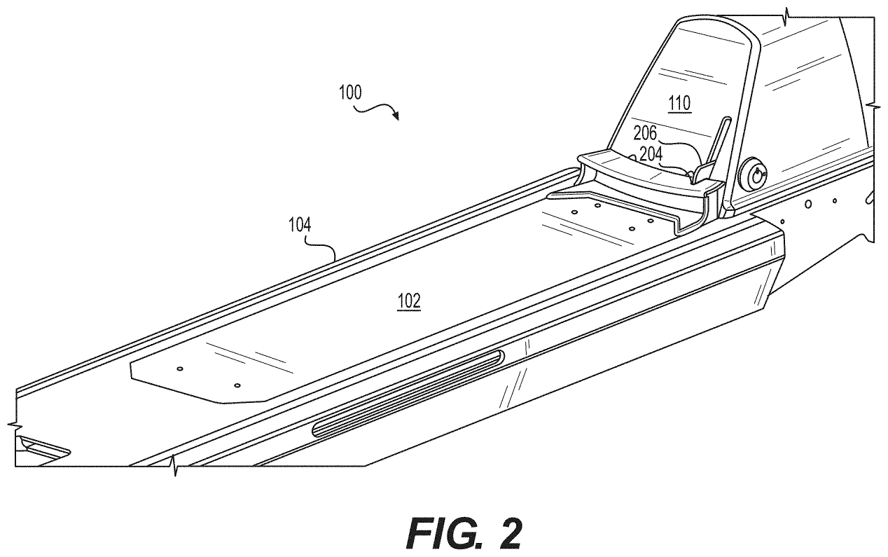

FIG. 2 illustrates further detail of the electric scooter 100 of FIG. 1. Referring to FIG. 2, the removable deck assembly 102 is held flush with the frame 104 by a latch 206 when engaged in a notch 204. The latch 206 may be controlled by a latch mechanism (not shown) disposed within the housing 110.

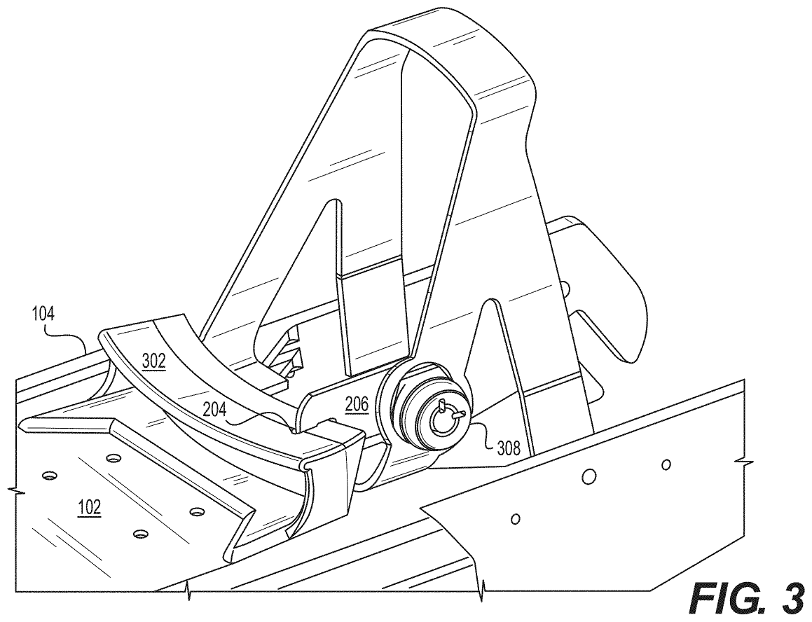

FIG. 3 illustrates further detail of the deck assembly 102 and latch 206 of FIGS. 1 and 2. Referring to FIG. 3, the deck assembly 102 may include a handle 302 to assist with the removal and installation of the deck assembly 102. The handle 302 may include a notch 204 to receive the latch 206. The deck assembly 102 may include a lock 308. The lock 308 may be operable to fix the latch 206 in an open position and/or a closed position, where the latch 206 secures the deck assembly 102 within the frame 104 when in the closed position. A key (not shown) may be inserted within lock assembly 308 to rotate the latch into, and out of, the notch 204, that is, between the closed position and an open position. When engaged with the notch 204, the latch retains the deck assembly 102 within the frame 104 of the scooter 100.

In the depicted embodiment, the lock assembly 308 is implemented as a physical lock, to be used with a physical key. But in other embodiments, the lock assembly 308 may be implemented in other ways. For example, the lock assembly 308 may be an electronic lock, which may be operated using an electronic key, fob, remote control, or the like. In embodiments where security is not required, the lock in the lock assembly 308 may be replaced with a knob, a button, or another mechanism. In any case, the lock assembly 308 may be hidden or disguised. This feature is especially useful in a ridesharing fleet, where users should not operate the lock assembly 308, or remove the deck assembly 102.

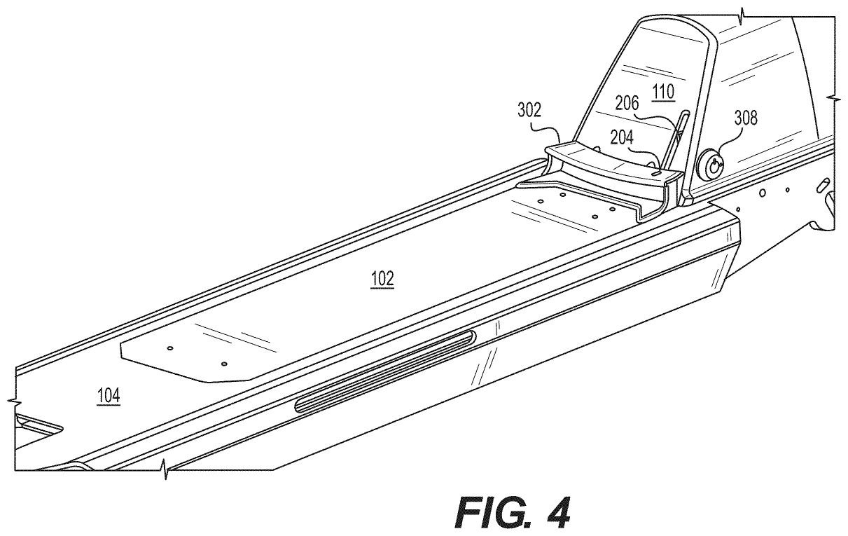

FIG. 4 illustrates detail of the scooter 100 of FIGS. 1 and 2 with the latch 206 in an open position. Referring to FIG. 4, the lock assembly 308 has been operated to rotate the latch 206 out of the notch 204. To protect the user from the latch, the latch 206 has been rotated to a position within the housing 110. The deck assembly 102 may now be removed from the scooter 100.

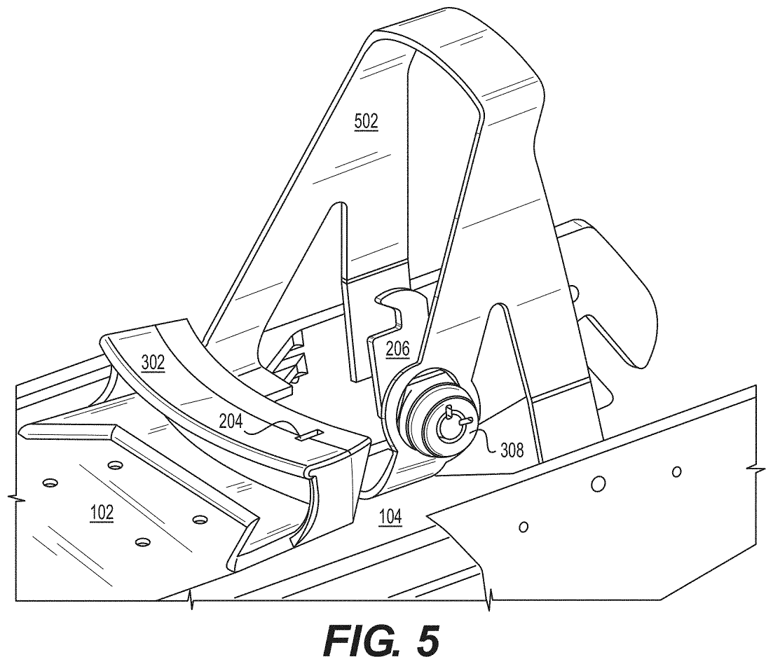

FIG. 5 illustrates further detail of the scooter 100 of FIGS. 1 and 2 with the latch 206 in an open position, and with the housing 110 removed. Referring to FIG. 5, the latch 206, and the lock assembly 308, are held in place by a strut 502 that is connected to the frame 104.

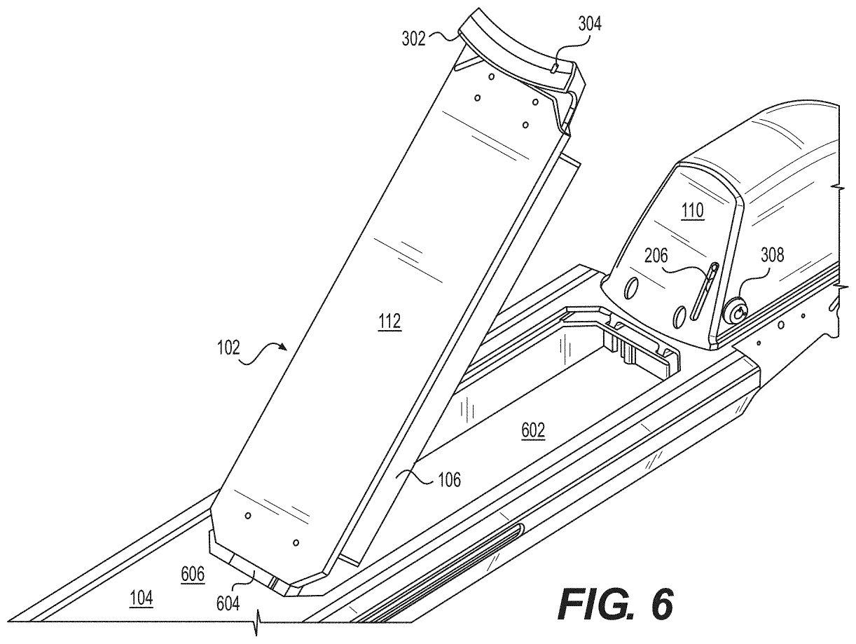

FIG. 6 illustrates detail of the scooter 100 of FIGS. 1 and 2 during installation, or removal, of the deck assembly 102. Referring to FIG. 6, the frame 104 has an upper surface 606, and an opening 602 in the frame 104 is visible. The opening 602 is formed so as to receive the deck assembly 102 when the deck assembly 102 is lowered into the opening of the frame from above the upper surface 606 of the frame. As can be seen in FIG. 6, the deck assembly 102 includes a protruding tongue 604 at the front of the deck assembly 102. During removal of the deck assembly 102, a user may lift the deck assembly 102 out of the opening of the frame 104 from above the upper surface 606 of the frame 104 by pivoting the deck assembly 102 upward about the tongue 604 using the handle 302, and then slide the deck assembly 102 slightly to the rear of the scooter 100 to disengage the tongue 604 from the frame 104. During installation of the deck assembly 102, a user may first insert the tongue 604 into the frame 104, pivot the deck assembly 102 downward into the opening 602 until flush with the frame 104, and then rotate the latch 206 into the notch 204 to secure the deck assembly 102 within the frame 104.

Also visible in FIG. 6 is the battery case 106. The battery case 106 may include one or more batteries (not shown), which may be cushioned with foam pads or similar materials. The battery case 106 may be integrated with the deck 112 to form the deck assembly 102, as noted above. The deck assembly 102 may be watertight to prevent damage to the batteries, and may be of automotive quality. This arrangement provides several advantages. In current designs, the battery case is mounted underneath a non-removable deck, for example using screws. In such designs, the batteries can only be removed by inverting the scooter, and unscrewing the battery case. During this process, the scooter may be damaged, the battery case may be damaged, and the screws may be lost. Furthermore, the user must have a tool such as a screwdriver. In contrast, in the described embodiments, the batteries may be removed without tools, by simply operating the latch 206 and lifting out the deck assembly 102. No tools are required. The scooter need not be inverted, and may remain on the ground, in a rack, or the like.

Other advantages are especially applicable to a fleet of shareable electric scooters. In current fleets, the scooters are generally collected each evening, and taken to a charging facility where the batteries are charged. The charged scooters are then returned to scooter sharing locations the next morning. But in this arrangement, the scooters are unavailable for sharing while being charged. And this arrangement requires two trips per day: one trip to collect the scooters, and another trip to deploy them.

Embodiments of the disclosed technology solve both of these problems. With the disclosed removable deck assembly, the scooters need not be collected. Instead, only the deck assemblies may be collected. The scooters may be left in the sharing location, sharing racks, and the like. Furthermore, with a fleet of similar scooters, the deck assemblies are interchangeable. Therefore, an operator can replace a discharged battery pack with a fresh battery pack, requiring only one trip, and keeping the scooter available while the discharged battery pack is recharged. And because the disclosed deck assemblies are much smaller than the scooters, many more scooters can be serviced by a single truck than with current arrangements. In addition, because the disclosed deck assemblies weigh less than the scooter, there is less likelihood an operator will be injured while lifting them.

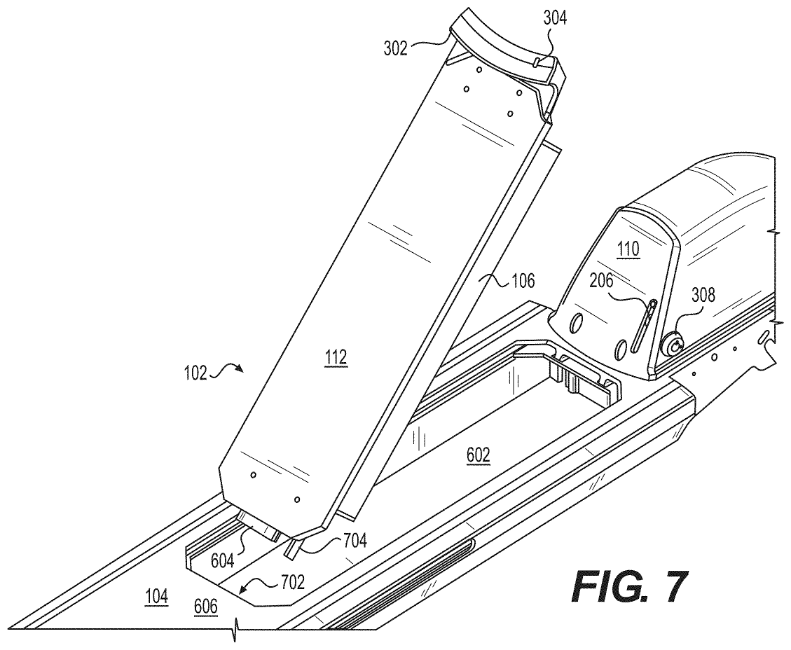

FIG. 7 illustrates further details of the scooter 100 of FIGS. 1 and 2 during installation, or removal, of the deck assembly 102 Referring to FIG. 7, the tongue 604 of the deck assembly 102 is free of the frame 104. As can be seen in FIG. 7, the frame 104 may feature a double-wall construction for rigidity and light weight. In the disclosed embodiment, a slot 702 may be formed between the walls of the frame 104 to receive the tongue 604. Also visible in the embodiment of FIG. 7 is a portion of an electrical power cable 704. The power cable 704 may provide power to the motor 108 of the scooter 100. To separate the deck assembly 102 from the scooter 100, the user may operate a connector of the power cable 704, as described in detail below.

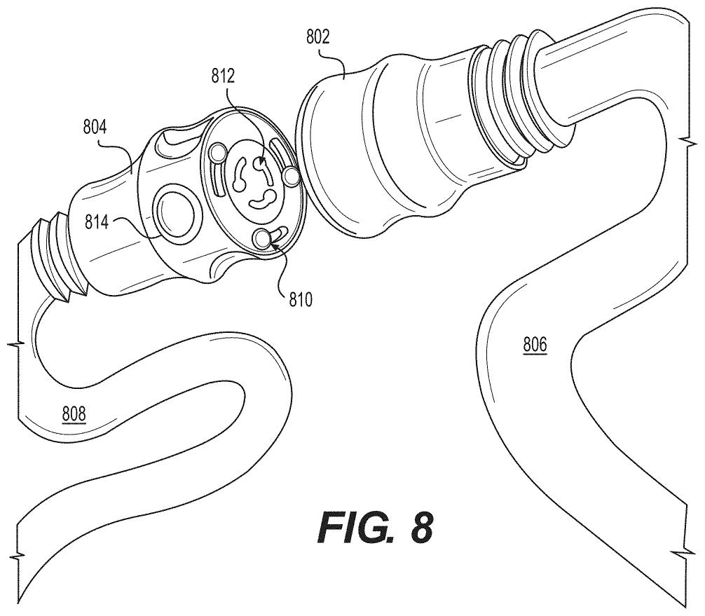

FIG. 8 illustrates a quick twist electrical soft connector according to embodiments of the disclosed technology. As used herein, the term “soft connector” is used to refer to a connector having two halves, where at least one of the halves is coupled to a flexible electrical cable. In some embodiments, the term “soft connector” is used to refer to a connector where both halves of the connector are coupled to respective flexible electrical cables. As described below, the flexible cable(s) serve to insulate the scooter from micro vibrations, a problem unique to vehicles such as scooters that have small, hard wheels. Referring to FIG. 8, the soft electrical connector includes a male half 802 and a female half 804. The halves 802, 804 are formed at the ends of electrical cables 806, 808, respectively. The illustrated soft connector is a quick twist connector that is opened and closed by twisting its halves 802, 804 in opposite directions. Accordingly, the female half 804 of the soft connector includes a plurality of curved slots 810, each including a round opening to receive a respective locking pin (not shown) of the male half 802. The electrical connectors may be implemented in a similar manner, as shown at 812.

In some embodiments, one half of the soft connector may include a locking indicator 814. The locking indicator 814 may shine red until the soft connector is completely closed, whereupon the indicator 814 may switch to green to indicate a positive lock of the soft connector.

One advantage of the disclosed quick twist electrical soft connector is that it mitigates the problem of micro vibrations. Vehicles such as automobiles and s are subject to vibrations caused by imperfections in the road surface. Vehicles with small, hard wheels, such scooters, are subject to these vibrations, and also to micro vibrations, which are caused by tiny imperfections in the road surface, for example such as the pebbles in a conglomerate road surface. Electrical connectors in particular are adversely affected by micro vibrations, which cause the mating electrical parts to rub together and thereby deteriorate. Gold plating on electrical connectors is particularly subject to this deterioration. In the disclosed embodiments, the lengths of electrical cables 806, 808 isolate the electrical connector from these micro vibrations, greatly reducing any wear the electrical connectors 812 experience.

Another advantage of the disclosed quick twist electrical soft connector is that it encourages users not to pull on the cables 806, 808 to open the soft connector. In conventional electrical connectors with no twist lock mechanism, users may be tempted to pull on the cables to open the connector. This abuse may shorten the life of the electrical cable and electrical connector considerably. But this is not possible with the twist connector. The user must grasp the soft connector halves in order to twist them in opposite directions. Consequently, the electrical soft connector and electrical cables 806, 808 may enjoy a longer lifespan.

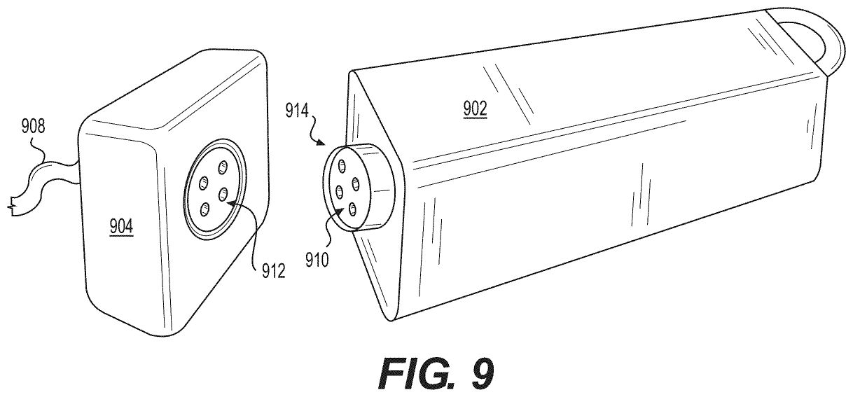

FIG. 9 illustrates a cushioned electrical connector according to embodiments of the disclosed technology. Referring to FIG. 9, a deck assembly 902 that includes a battery pack may be pressed against an elastic mounting block 904 during installation. The deck assembly 902, and the mounting block 904, include respective electrical connectors 910, 912 that are mated during installation of the deck assembly 902, thereby providing power from the battery pack to the motor through an electrical power cable 908. The mounting block 904 may be fabricated of an elastic material such as rubber to cushion the electrical connectors 910, 912 from micro vibrations. In the embodiment of FIG. 9, the elastic mounting block 904 is disposed upon the scooter.

But in other embodiments, an elastic mounting block may be disposed on the deck assembly 902 instead, or as well. For example, as shown in FIG. 9, the deck assembly 902 may include a second elastic mounting block 914 to further isolate the electrical connectors 910, 912 from micro vibrations. These elastic mounting blocks 904, 914 may also form a seal about the electrical connectors 910, 912 that protects the electrical connectors 910, 912 from water, dirt, and the like.

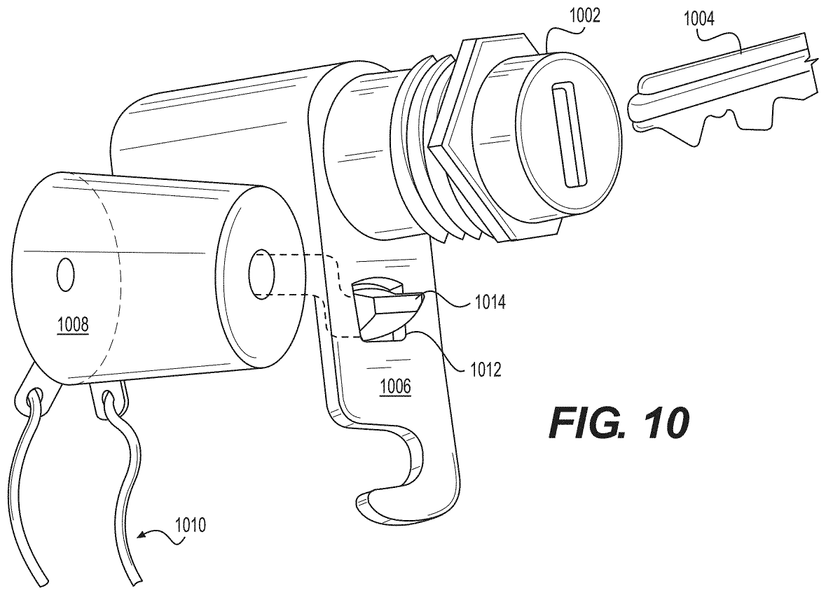

FIG. 10 illustrates a compound locking assembly according to embodiments of the disclosed technology. Referring to FIG. 10, the compound locking assembly includes a mechanical lock 1002, which may be operated by a physical key 1004 to rotate a latch 1006 into a corresponding notch, such as notch 204 in handle 302 of deck assembly 102, as shown in FIG. 3.

Referring again to FIG. 10, the compound locking assembly may also include an electric lock 1008, which may receive power through electrical cables 1010, and which may be operated using an electronic key, fob, remote control, or the like. When operated, the electric lock 1008 may insert a tab 1014 into an opening 1012 formed in the latch 1006 of the mechanical lock 1002, thereby preventing operation of the mechanical lock 1002.

In some embodiments, the electric lock 1008 may operate in parallel with the mechanical lock 1002. In such embodiments, the electric lock 1008 may insert the tab 1014 into a notch in the deck assembly. In such embodiments, both locks 1002, 1008 must be opened to release the deck assembly.

In some embodiments, the tab 1014 of the electrical lock 1008 may have multiple stops. In one of the stops, the tab 1014 engages the latch 1006 of the mechanical lock 1002, thereby preventing its operation, as illustrated in FIG. 10. In another of the stops, the tab 1014 engages a notch in the deck, thereby preventing its removal, as described above. In still another one of the stops, the tab 1014 engages neither the latch 1006 nor the deck assembly, thereby permitting operation of the mechanical lock 1002, and removal of the deck assembly.

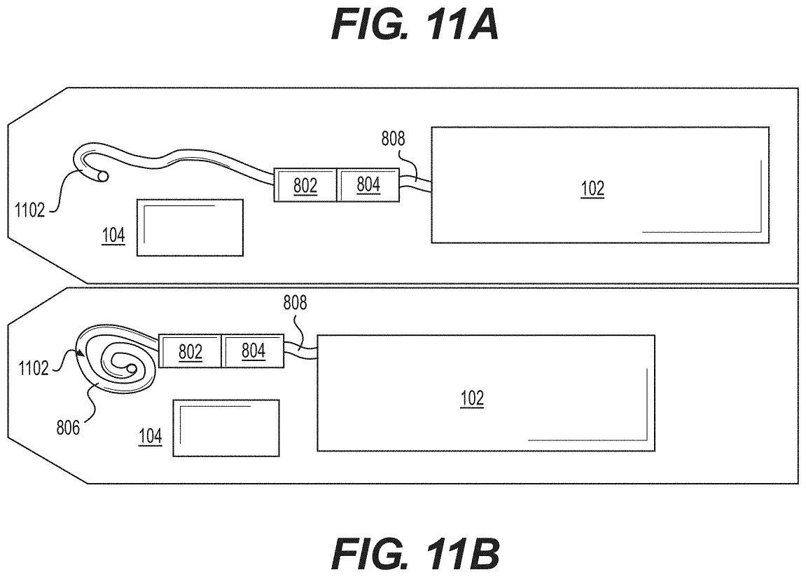

In embodiments that include an electrical power cable, the scooter may include a mechanism to retain and protect the cable when the deck assembly is installed. FIGS. 11A, B illustrate one such mechanism according to embodiments of the disclosed technology. In FIGS. 11A, B the mechanism is illustrated for the electrical cables 806, 808 and electrical connector 802, 804 of FIG. 8. However, the mechanism may be employed with any electrical cable and electrical connectors.

FIGS. 11A-B are top views of the scooter, with the rear of the scooter at the left. FIG. 11A illustrates a portion of the scooter 100 during removal or installation of the deck assembly 102. The battery pack in the deck assembly 102 is electrically coupled to the motor 108 by the electrical cables 806, 808 and the electrical connectors 802, 804. As shown in FIG. 11A, during installation or removal of the deck assembly 102, one or both of the electrical cables 806, 808 are extended to facilitate installation and removal, and to provide easy access to the electrical connectors 802, 804. A retention device 1102 permits this extension of the electrical cables 806, 808.

When the deck assembly 102 is installed in the frame 104 of the scooter 100, the retention device 1102 retracts, guides, organizes, and stores the loose portions of the electrical cables 806, 808, as shown in FIG. 11B. For example, the electrical cables 806, 808 may be retracted into a channel (not shown) formed in the frame 104 of the scooter 100. The retention device 1102 may be implemented as a spring-loaded device, for example such as a winding mechanism or the like. The winding mechanism may be similar to that used in spring-loaded tape measures, with the electrical cables 806, 808 taking the place of the tape. One benefit of this mechanism is that a technician working on the scooter does not have to manually feedback the slack in the electrical cables 806, 808, that results from the removal of the battery pack. When retracted, the electrical cables 806, 808, and the electrical connectors 802, 804, are protected from pinching, wear, and the like.

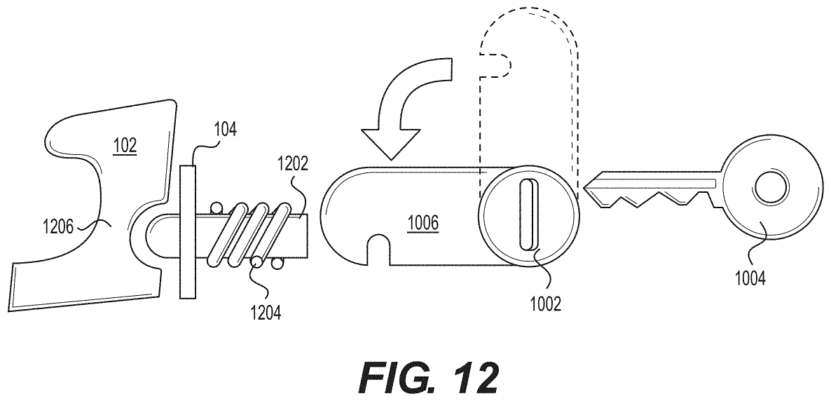

In some embodiments, the latch that retains the deck assembly 102 within the frame 104 of the scooter 100 may be hidden within a structure such as the frame 104 or the housing 110 of the scooter 100 so that it cannot be seen, and to protect the latch from damage. One such embodiment is illustrated in FIG. 12. The embodiment of FIG. 12 is illustrated for the mechanical lock 1002, physical key 1004, and latch 1006 of FIG. 10. However, the described embodiment may be employed with any lock, key, and latch, or with a keyless latch where the lock and key are replaced by a knob or the like.

Referring to FIG. 12, the described embodiment also includes a pin 1202 and a spring 1204 that biases the pin 1202 against the frame 104. When the lock 1002 and key 1004 are used to rotate the latch 1006 downward into a locked position, the latch 1006 forces the pin 1202 through a hole in the frame 104 into a notch 1206 formed in the deck assembly 102, thereby retaining the deck assembly 102 within the frame 104. When the lock 1002 and key 1004 are used to rotate the latch 1006 upward into an unlocked position, the spring 1204 backs the pin 1202 out of the notch 1206 so the deck assembly may be removed.

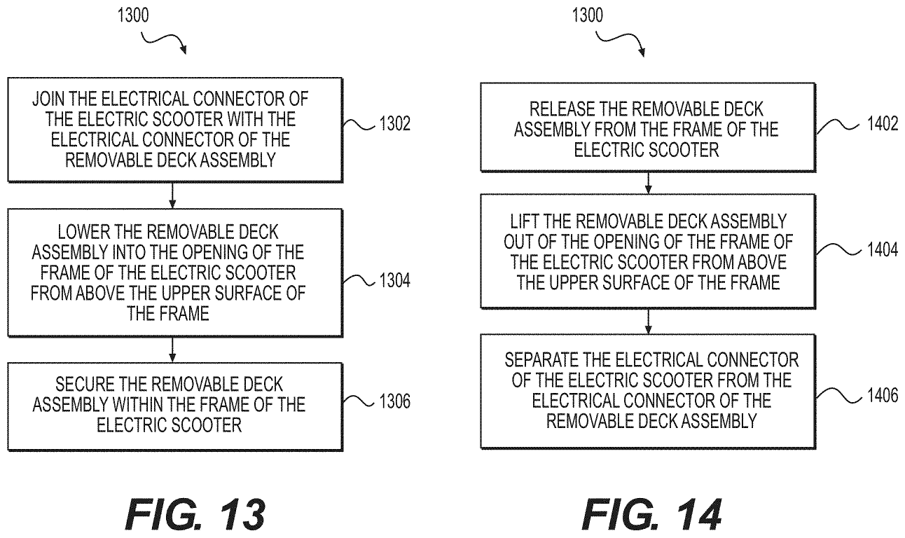

FIG. 13 illustrates a process 1300 for a user to install a removable deck assembly into an electric scooter according to embodiments of the disclosed technology. While elements of the process 1300 are described in a particular sequence. It should be understood that certain elements of the process 1300 may be performed in other sequences, may be performed concurrently, may be omitted, or any combination thereof.

Referring to FIG. 13, the user may join the electrical connector of the electric scooter with the electrical connector of the removable deck assembly, at 1302. The connectors may be joined as described above. The user may lower the removable deck assembly into the opening of the frame of the electric scooter from above the upper surface of the frame, at 1304, for example as described above. The user may secure the removable deck assembly within the frame of the electric scooter, at 1306, for example as described above.

FIG. 14 illustrates a process 1400 for a user to remove a removable deck assembly from an electric scooter according to embodiments of the disclosed technology. While elements of the process 1400 are described in a particular sequence, it should be understood that certain elements of the process 1400 may be performed in other sequences, may be performed concurrently, may be omitted, or any combination thereof.

Referring to FIG. 14, the user may release the removable deck assembly from the frame of the electric scooter, at 1402, for example as described above. The user may lift the removable deck assembly out of the opening of the frame of the electric scooter from above the upper surface of the frame, at 1404, for example as described above. The user may separate the electrical connector of the electric scooter from the electrical connector of the removable deck assembly, at 1406 for example as described above. Spatially relative terms such as “under,” “below,” “lower,” “over,” “upper,” and the like, are used for ease of description to explain the positioning of one element relative to a second element. These terms are intended to encompass different orientations of the device in addition to different orientations than those depicted in the Figures. Further, terms such as “first,” “second,” and the like, are also used to describe various elements, regions, sections, etc. and are also not intended to be limiting. Like terms refer to like elements throughout the description.

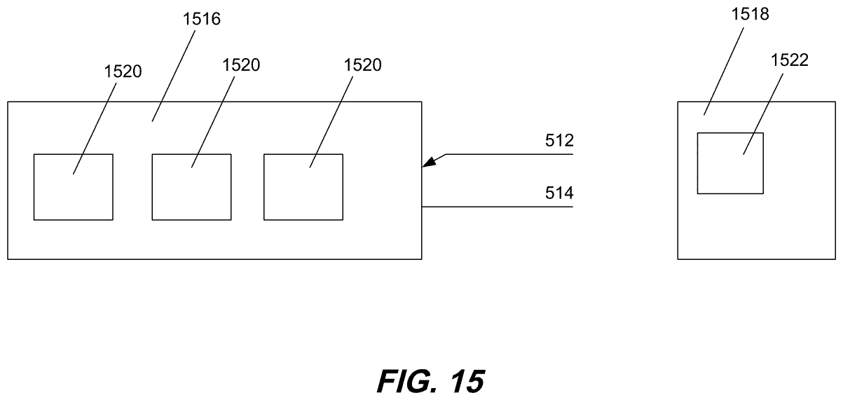

In one embodiment, illustrated in FIG. 15, electric vehicles 1516 are provided with systems and methods for vehicle security without a hardware secure element 1510. Hardware secure elements 1510 usually allow for the storage of private keys 1512, which are used to sign and encrypt data. In one embodiment the present invention removes the dependency on a hardware secure element 1510 as part of the whole security system.

Private keys and private key pairs (collectively 1512 and 1514) are used to cryptographically secure sensitive information, private keys 1512 can be used to decrypt, encrypt, or sign data, the corresponding public key 1514 can be used to decrypt or verify the signature of the data signed by its private key, public keys cannot be used to encrypt or sign data.

As a non-limited example, as used herein a vehicle 1516 is a means of carrying or transporting something including but not limited to an EV motor vehicle 1516, including but not limited to a scooter, skateboard, skates, and the like.

As used herein an encryption key is a piece of information that determines the functional output of a cryptographic algorithm. For encryption algorithms, a key specifies the transformation of plaintext into ciphertext, and vice versa for decryption algorithms. Keys also specify transformations in other cryptographic algorithms, such as digital signature schemes and message authentication codes.

As used herein, the cloud 1518 is a global network of servers, each with a unique function. There is not a physical entity, but instead is a vast network of remote servers around the globe which are hooked together and meant to operate as a single ecosystem. These servers are designed to either store and manage data, run applications, or deliver content or a service such as streaming videos, web mail, office productivity software, or social media. Instead of accessing files and data from a local or personal computer, you are accessing them online from any internet-capable device—the information will be available anywhere you go and anytime you need it. In the case of this embodiment the cloud 1518 is securely storing and generating public key and private key pairs for each component in the vehicle 1516.

As non-limiting examples, there are four different methods to deploy 8 resources.

These include: a public cloud 1518 that shares resources and offers services to the public over the Internet; a private cloud that isn't shared and offers services over a private internal network typically hosted on-premises; a hybrid cloud that shares services between public and private clouds depending on their purpose; and a community cloud 1518 that shares resources only between organizations, such as with government institutions.

In one embodiment, system 10 is coupled to the cloud 1518.

As used herein, a local area network (LAN) is a network that interconnects within a limited area such as a residence, school, laboratory, university campus or office building. By contrast, a wide area network (WAN) not only covers a larger geographic distance, but also generally involves leased telecommunication circuits. Ethernet and Wi-Fi are two common technologies in use for local area networks. Historical network technologies include ARCNET, Token ring, and AppleTalk.

As a non-limiting example, a wide area network (WAN) is a network that exists over a large-scale geographical area. A WAN connects different smaller networks, including local area networks (LANs) and metro area networks (MANs). This ensures that computers and users in one location can communicate with computers and users in other locations. WAN implementation can be done either with the help of the public transmission system or a private network.

As a non-limiting example, system 10 is coupled to the cloud. This can be achieved via GSM, WiFi, satellite, a mobile device and the like.

Other wireless standards that are specifically designed for IoT devices are becoming available such as Lora, NB-IOT and LTE-M, and the like.

As a non-limiting example, in one embodiment one or more hardware elements 1510 of the vehicle 1516 has public keys 1514 stored therein. Secure encryption is not put on the hardware elements 1510.

A vehicle 1516 consists of one or more in individual components 1520. Individual components 1520 of the vehicle 1516 are given an Acton Unique Identifier (AUIDs). When a vehicle 1516 is activated the first time, a unique public key 1514 and private key 1512 pair are generated by the cloud. AUIDs, public key and private keys 1514 and 1512 are then stored in the cloud. Each component stores its AUID and public key in persistent memory within the component thus eliminating theft of private keys 1512.

For selected components 1520 of the vehicle 1516, the cloud 1518 produces a unique private key 1512 and a public key 1514. As a non-limiting example, with the present invention, private keys 15112 are secure and in the cloud. They cannot be taken from the vehicle 1516. Non-limiting examples of vehicle 1516 components 1520 with public keys 1514 include but are not limited to: IOTA, the battery, motor controller, and the like.

As non-limiting examples, a simple electric vehicle 1516 can include a battery; vehicle control unit (motor controller), and IoT gateway. Each of these components 1520 is given an AUID. Additional components 1520 include but are not limited to vehicle locks; dashboards; helmets; docking stations; and the like.

As non-limiting examples, selected vehicle components 1520 have unique IDs with a unique identifier. These components 1520 are given a unique key pair. As a non-limiting example, the private key 1512 is securely stored in the cloud. An associated public key 1512 is stored in the vehicle components 1520. Communication in the cloud 1518 can be authenticated with the vehicle 1516 through the components 1520 that have public keys.

As a non-limiting example of authentication steps, public keys 1514 are passed to the vehicle 1516, e.g., vehicle components 1520. The private key 1512 is stored in the cloud, and the public key 1514 is transferred to a respective vehicle component.

As a non-limiting example, when the vehicle 1516 connects to the server 1522, it tells the server 1522 it has components 1520 A, B, and C. The System looks up in an associated database and generates an activation message composed of multiple parts, each part signed with the private key 1512 that corresponds to the AUID of the vehicle component A, B, or C 1510. When the activation message is received by the vehicle 1516, the individual components 1520 A, B, and C will decrypt and verify their parts of the message. If anyone component's message part fails verification, the vehicle 1516 will not activate.

As a non-limiting example, a secret key is not needed that unlocks the entire scoter. Instead, the system creates components 1520 are identified as being unique with associated keys.

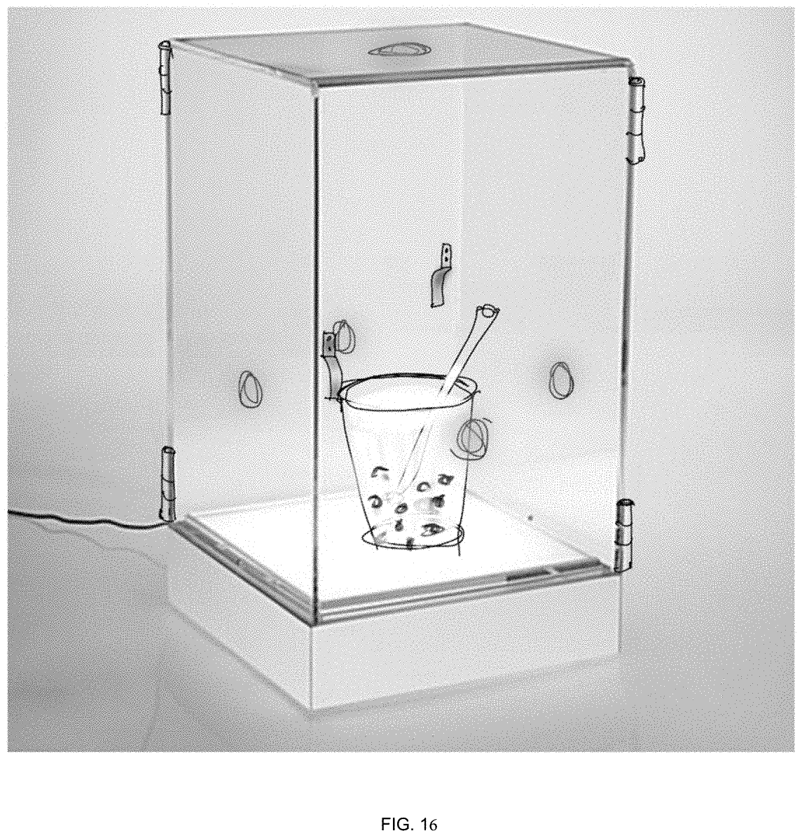

As illustrated in FIG. 16, in one embodiment fleets of vehicles are used to distribute information between vehicles in the fleet. As a non-limiting example, individual fleet vehicles have two wireless communication networks. The first is any kind of cloud 1518 connectively. The second one is any kind of local wireless communication.

When vehicles communicate with the cloud, they report their status occasionally. When they report status, they report the presence of other fleet-vehicles that they have detected on local wireless. As a non-limiting example, this status message can then be communicated with other fleet vehicles IDs that are within local communication. This provides information about the location of fleet vehicles, which can be used to reduce theft and increase fleet availability.

As a non-limiting example, data can be distributed to the fleet by seeding it to only certain vehicles, and these vehicles that receive the communications then communicate with other vehicles. Data that could be sent includes, but is not limited to updates, navigation information, vehicle configuration, secure one-time-keys. This mechanism decreases fleet-wide data-usage and improves fleet operation.

As a non-limiting example, a vehicle 1516 can detect, via local wireless communication, other vehicles, report their presence to the cloud, and the can then determine if another vehicle 1516 is located within a selected proximity. The cloud 1518 can then determine if the reporting vehicle 1516 can communicate data to the other vehicle. The cloud 1518 can then send a one-time use session key to the vehicles, allowing them to communicate securely.

When a vehicle 1516 communicates with the cloud 1518 that it sees another vehicle, it sends this message up to the cloud. The cloud 1518 can use this vehicle 1516 presence information to disable vehicles, track stolen vehicles, locate missing vehicles, and the like.

Fleet vehicles are vehicles operated by an entity that provides them for public or private use to individuals or employees. A fleet is a group of one or more Fleet Vehicles that an operator makes available for use. Private vehicles are vehicles operated by individuals for their own use.

In one embodiment, this invention can be used with both fleet and individual vehicles. If individual or fleet Operators of EV include their vehicle 1516 in this system, the benefits of lost vehicle 1516 discovery, reduced data usage, and the like can be extended across fleets and individuals. In this way, the fleet vehicles of Operator A can look for a stolen fleet vehicle 1516 of Operator B, while a private vehicle 1516 operated by individual C can receive software update data from Operator A's fleet.

When misplaced or stolen fleet or individual vehicles are located, the owner and/or authorities can be notified.

Virus pose a problem for all. The coronavirus includes but is not limited to the following: 229E (alpha); NL63 (alpha); 0C43 (beta); HKU1 (beta); MERS-CoV; SARS-CoV; SARS-CoV-2; and the like.

As non-limiting examples, UVC lights, 200-400 nm, can be provided, as well as any light that kills pathogens, including but not limited to light with intensity for a certain amount of time, lights that shine on the handlebar, or a plastic contact surface that is made with lights at interior.

In one embodiment, energy is supplied to the surface to equal to or exceed 60 degrees F.

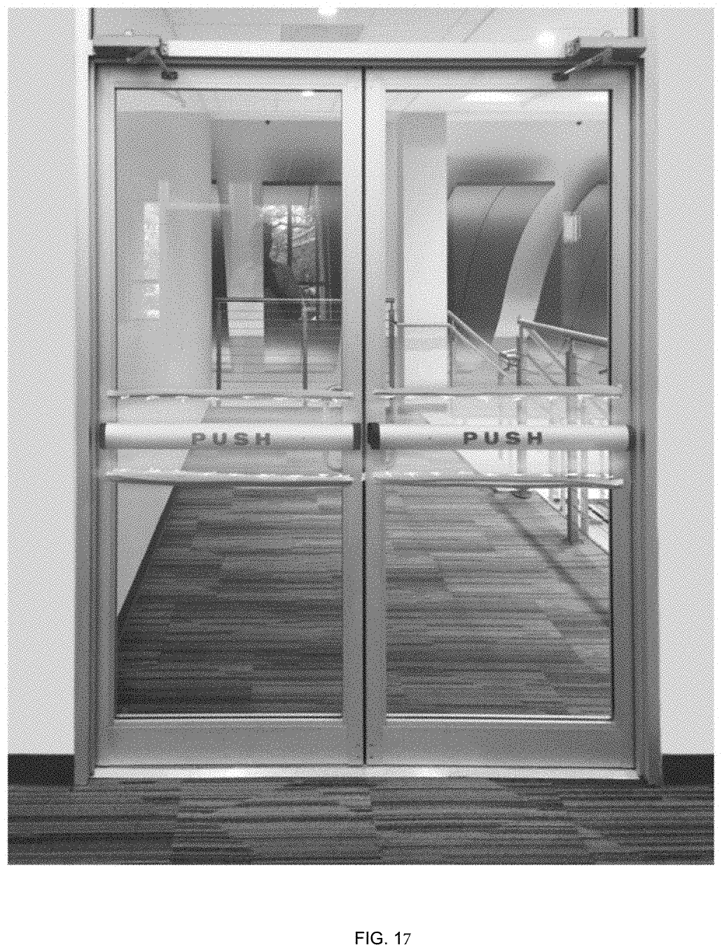

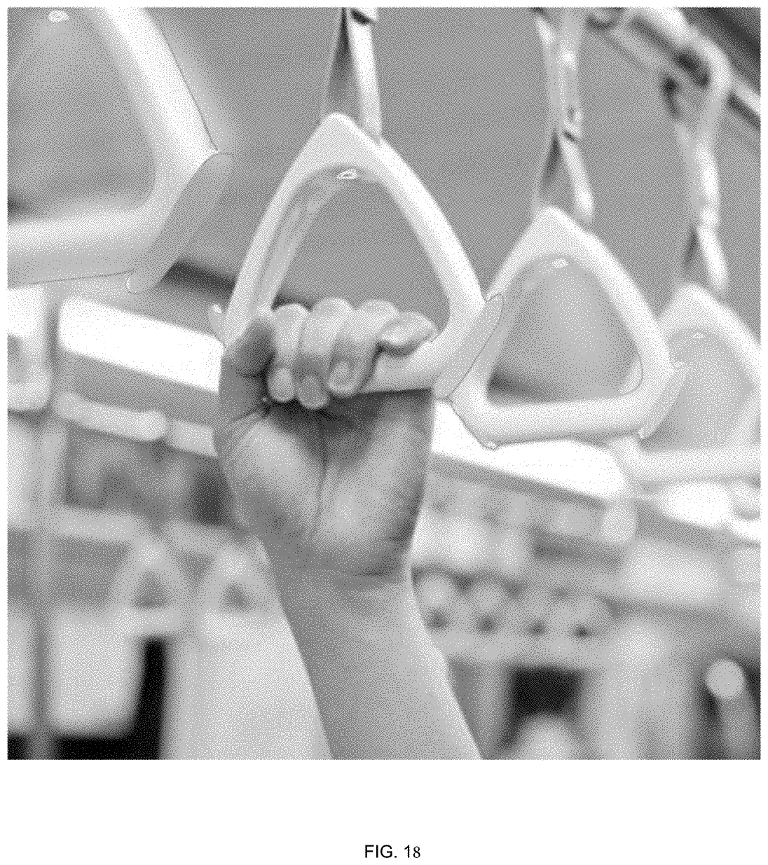

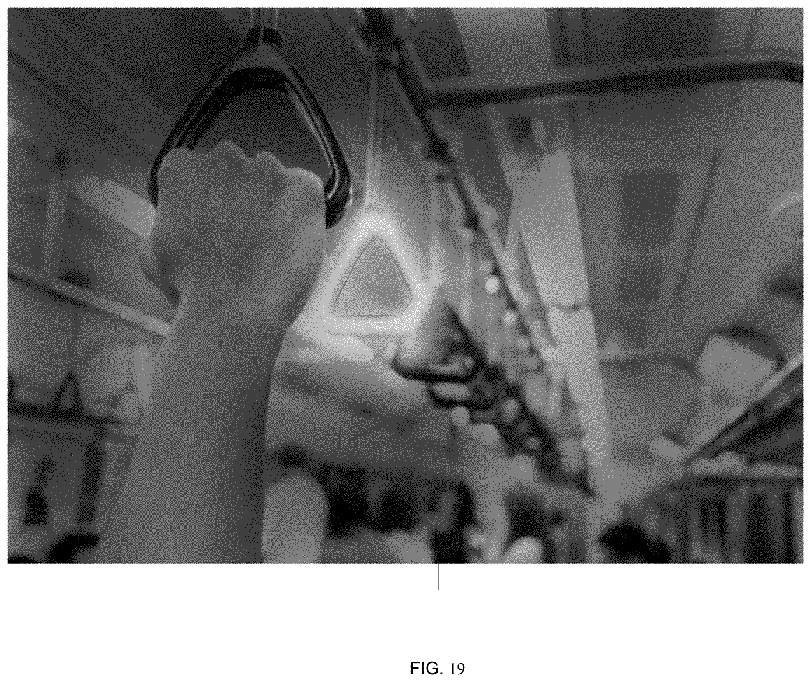

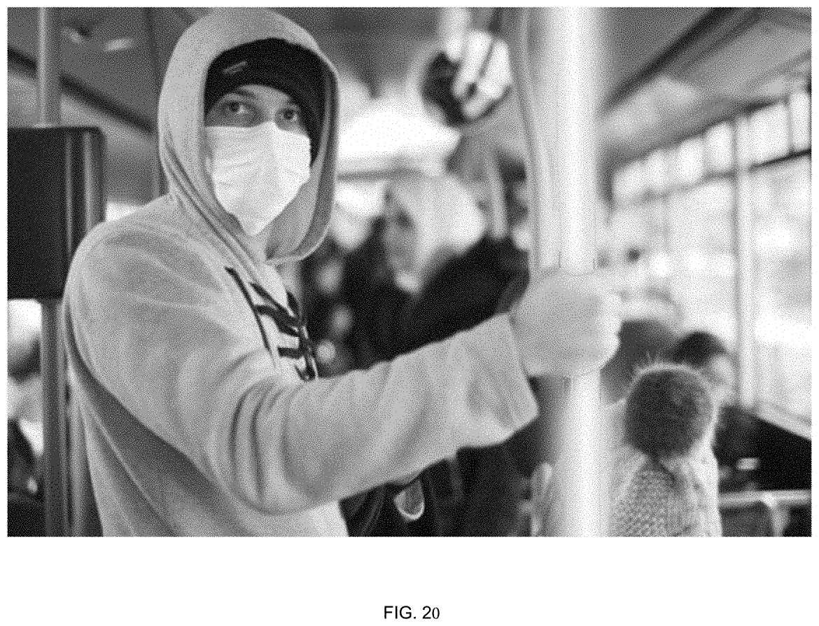

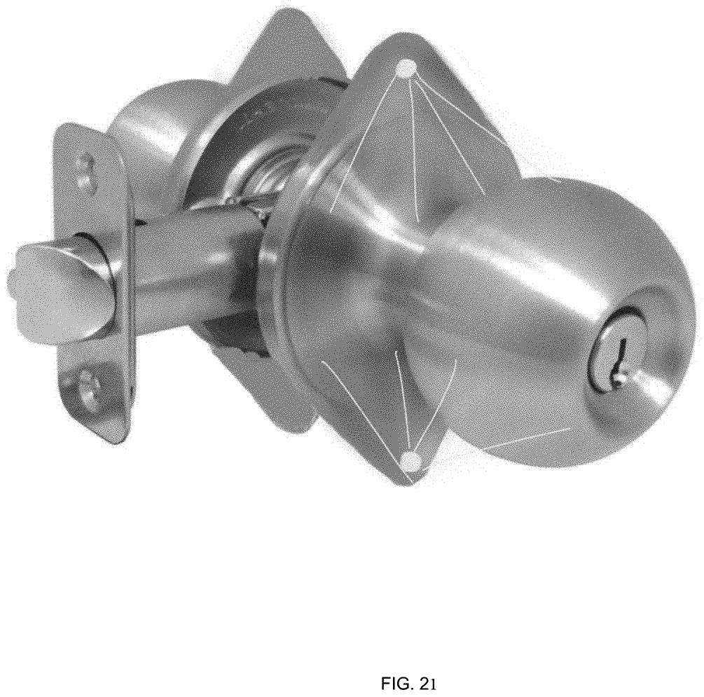

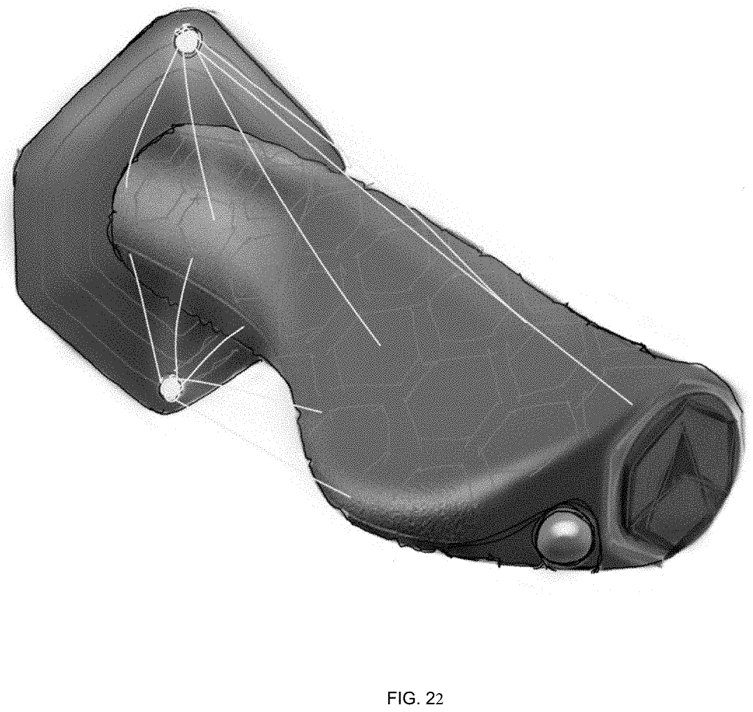

Referring to FIGS. 17-24 UVC can be used for different surface to kill the coronavirus, in one embodiment any surface of a vehicle, included but not limited to bicycles, electric vehicle, non-electric vehicle, and the like, that an operator or rider is in contact with, including but not limited to handlebars, as well as any surfaces that more than one-person use, including but not limited to: public transport surfaces, airline surfaces, bus handles, car handles, knobs, can all include one or more of: a light source, disinefficient or the like, is provided that kills the coronavirus.

In one embodiment any type of handle device that people touch, externally with UV, or internally with the light shining the inside.

As non-limiting examples, door knobs include but are not limited to: entrance door handles typically used on exterior doors, and include keyed cylinders; privacy door handles typically used on bedrooms and bathrooms that can be locked; and passageways with doors such as halls and closets.

As non-limiting examples, any type of handle device that people touch, externally with UVC, or internally with the shining inside.

In one embodiment, UVC can be used with buttons touched by people. These buttons are difficult to avoid, which is part of the reason why push buttons can be crawling with germs.

Further, ubiquitous buttons, are found on ATMs, elevators, telephones and drink machines, among other things, are located in areas that are not often cleaned and disinfected to kill bacteria and viruses.

In another embodiment, any surface of a vehicle that an operator or rider is in contact with can be made from an antibacterial/antimicrobial plastic (hereafter “sanitizing layer/coating”), or an associated exterior surface, where the operator or rider contacts, including but not limited to handle bars, grips, brake levers, and the like, can be treated with an antibacterial/antimicrobial material (hereafter “contact surface”).

“). As a non-limiting example, an actual paint, applied at the factory, can include these elements.

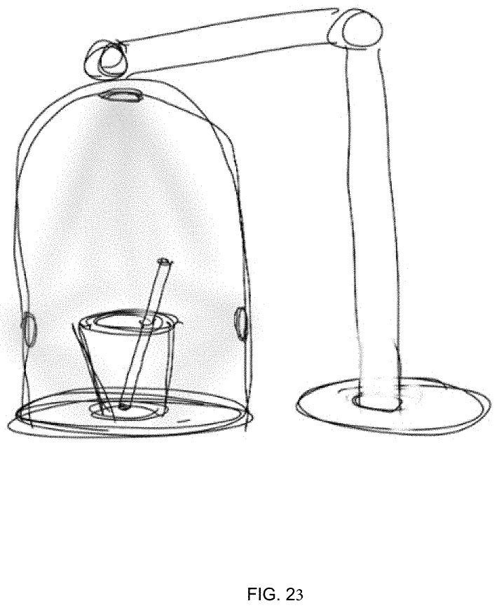

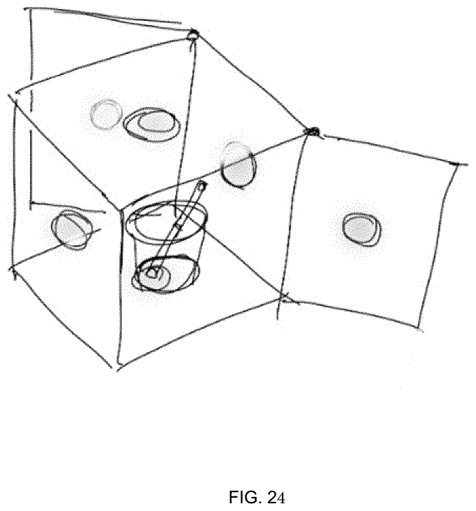

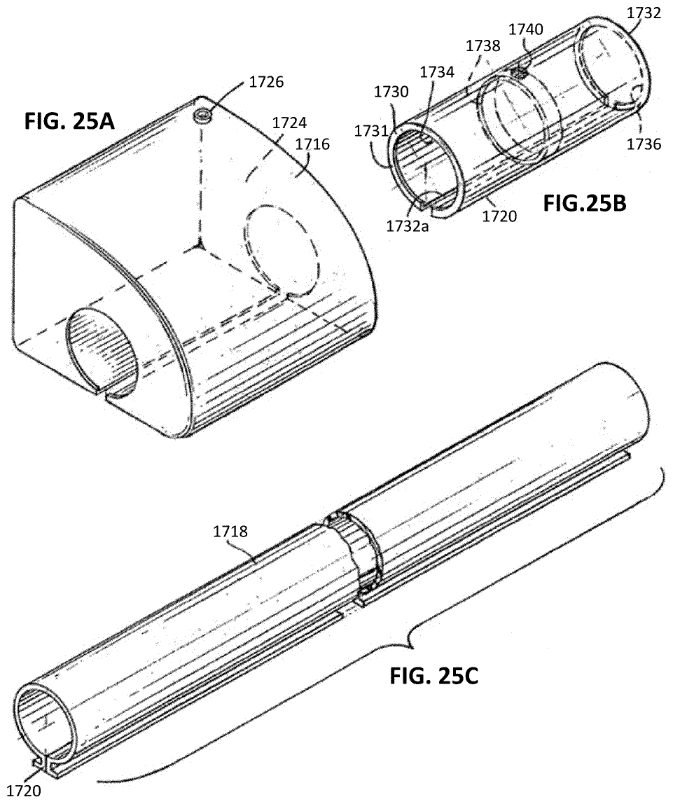

FIGS. 25(a) through 25(c) depict three primary components of an applicator device 1700 for a sanitizing layer/coating, has a main housing 16 shown in FIG. 25(a), a sanitizing layer/coating dispensing and spreading tube 1728 shown in FIG. 4B and replacement cover and rail mechanism 1718 shown in FIG. 25(c). The main housing 16 fits over and cooperatively engages the dispensing and spreading tube 1728, with both the dispensing and spreading tube 1728 and the housing 1716 being mounted on the replacement cover and rail mechanism 1718.

The main housing 1716 shown in FIG. 25(a) is comprised of a molded plastic housing 1716 with an internal reservoir 1724 which may hold liquid, including, for example, an anti-bacterial liquid. The main housing 1716 also contains a reservoir refill valve 1726 shown on the top right hand rear corner through which the reservoir 1724 may be refilled using a nozzle or filling tube of appropriate dimensions.

The dispensing and spreading tube 1728 can include a partial cylindrical tube 30 open on both ends 1731, 1732 and having a slot 1732A along its entire length.

In one embodiment, spreading tube 1728 is provided and can be made of a felt material or similar porous material which will permit liquid from the reservoir 1724 within the main housing 1716 to be wicked and ultimately make contact with the replacement cover and rail mechanism 1718.

The dispensing and spreading tube 1728 can include one or more apertures 1740 for flow of sanitizing layer/coating from reservoir 1724.

In one embodiment, a piston and cylinder pump mechanism 1725 is used to move liquid from reservoir 1724 through tube 1727 into tube 1731 and onto central spreader 1736. Liquid pumped from reservoir 1724 is then spread along the length of the desired surface

In one embodiment, the pump mechanism 1725 is activated by pushing push button actuator 1733 Each push of push button actuator 1733 causes the cylinder of pump mechanism 1725 to depress thereby transferring a predetermined amount of liquid onto replacement cover and rail mechanism 1718.

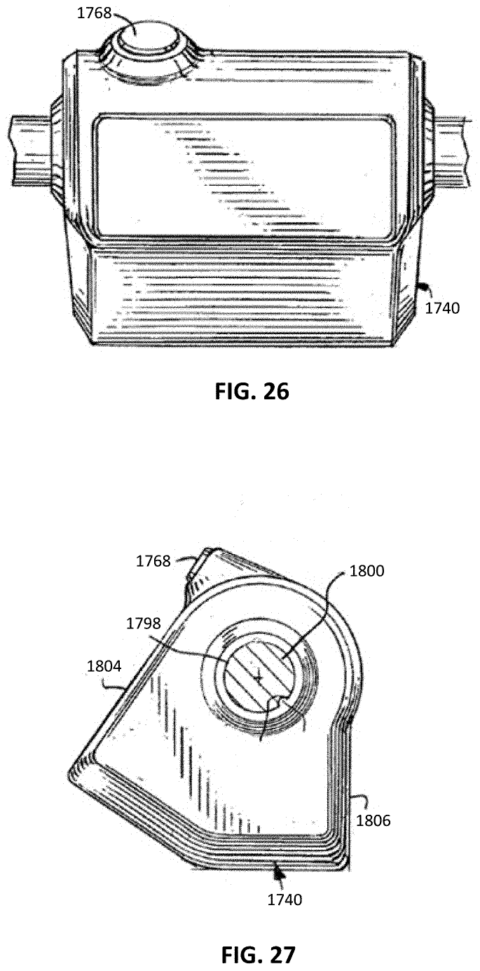

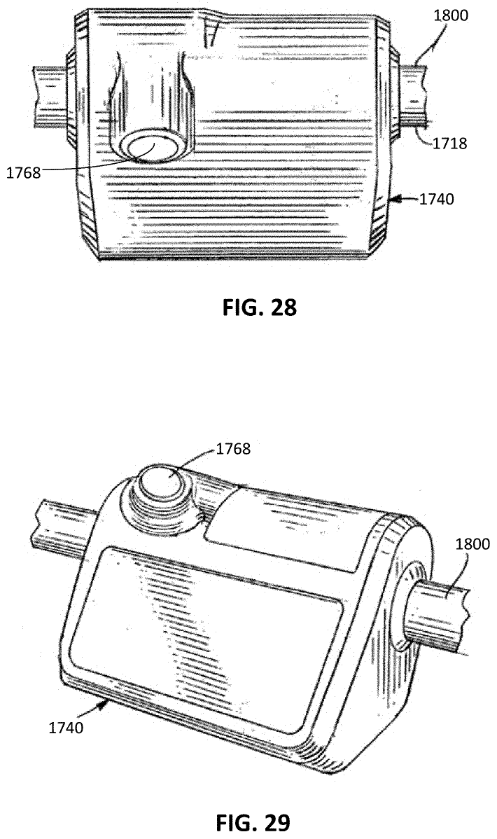

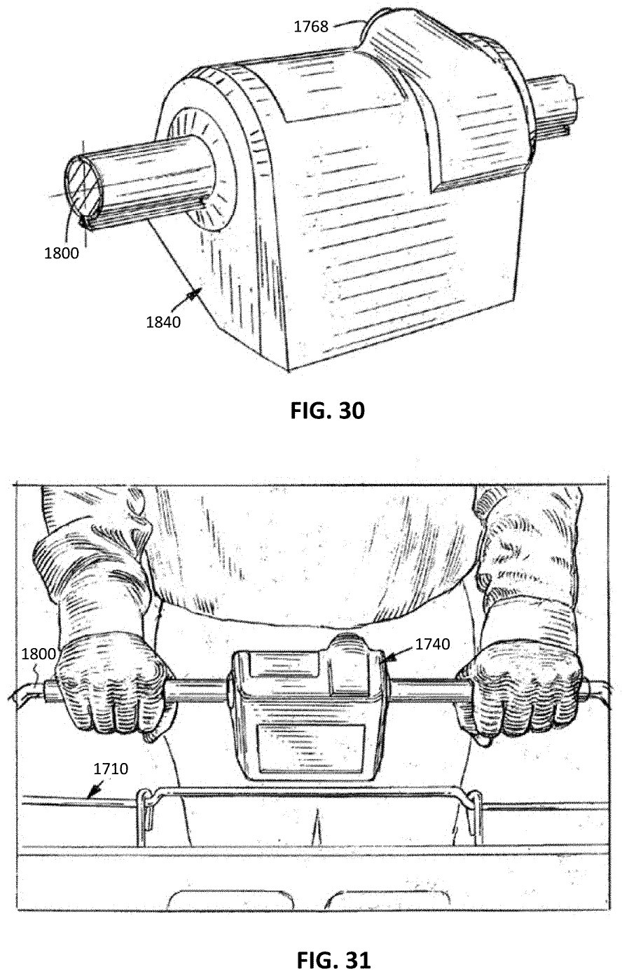

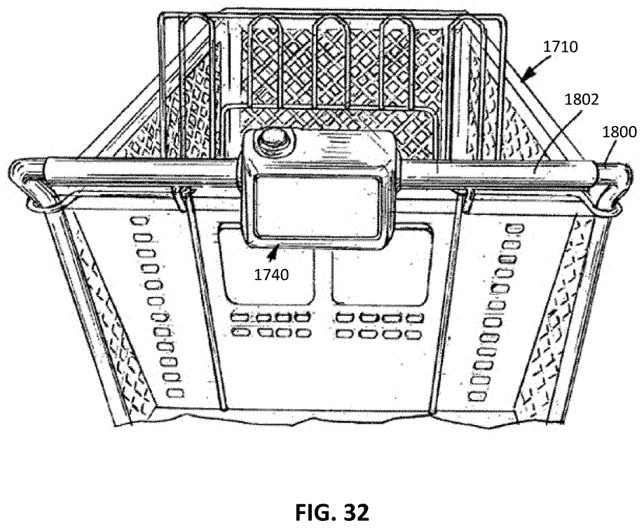

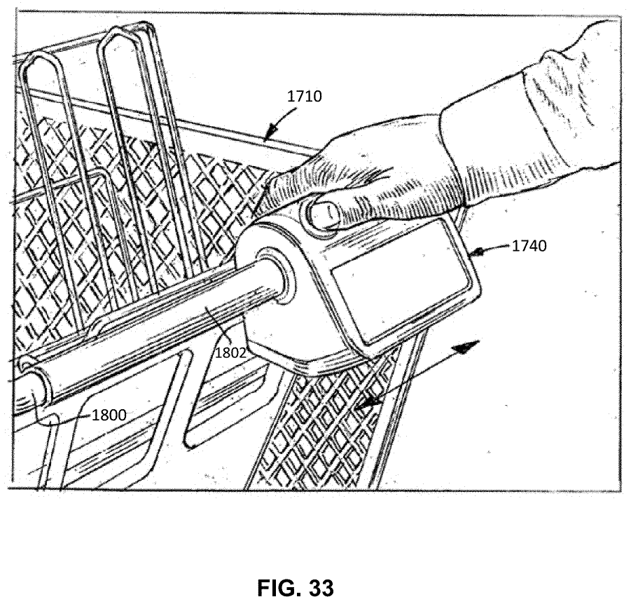

FIGS. 26 through 33 illustrate one embodiment where reservoir 1724 positioned within the chamber 1746 is a reservoir in the form of a bottle 1748. As a non-limiting example, the bottle 1748 can include a removable cap 1750 so that sanitizing layer/coating s can be filled into the bottle 1748.

As a non-limiting example, a conduit 1764 connects from the bottle reservoir 1748 through the cap 1750 to a pump mechanism 1766. The pump mechanism 1766 is operated by a push button 1768 which is manipulated to pump sanitizing layer/coating through conduits, such as conduit 1770, and a secondary conduit 1772. In one embodiment, the reservoir 1748 includes a vent 1776 which permits air inflow into the reservoir 1748 as sanitizing layer/coating is pumped therefrom.

In one embodiment, gravity feeding or wick feeding can be used for sanitizing layer/coating.

In one embodiment a housing 1820 containing a reservoir 1821 is maintained generally in a fixed location relative to a surface and a reservoir applicator 1822 docks with the reservoir. As a non-limiting example, the housing 1820 can include a fill port 1825A, by means of which the reservoir 1821 can be re-filled with sanitizing layer/coating. The fill port 1825A may be provided with a cap or stopper to prevent unwanted spillage of the sanitizing layer/coating, or with a one-way reservoir refill valve.

The housing 1820 can include a push button 1823 that can be activated by a use to dispense (apply) sanitizing layer/coating to the reservoir applicator. As a non-limiting example, the push button 123 may be linked to a pump mechanism of a similar type to the pump mechanisms 1725.

In one embodiment, the housing 1820 is snap-fitted around the surface of the electric vehicle 1800, including but not limited to a handle bar 1800 and fastened to the handle bar 1800 using tamper resistant fasteners 1820A, 1820B and a bar 1821A, to maintain the reservoir 1821 at a fixed location of the handle bar.

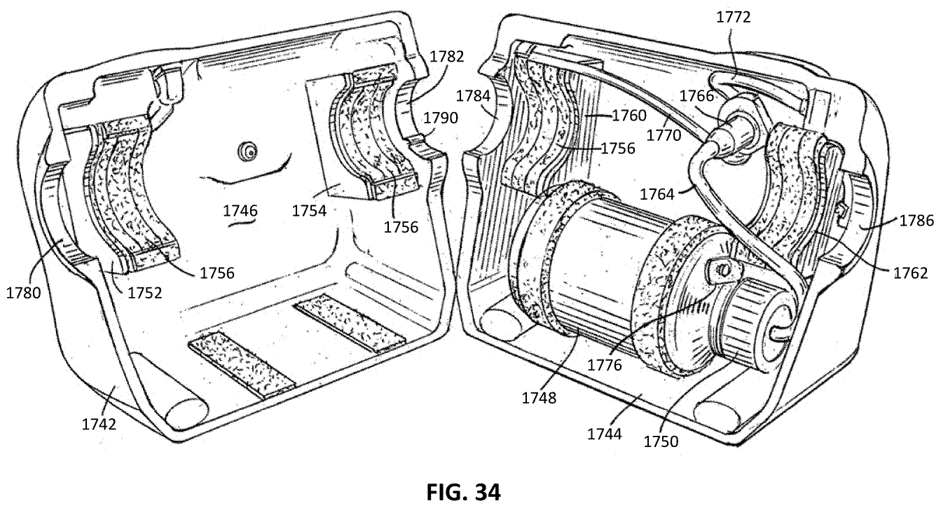

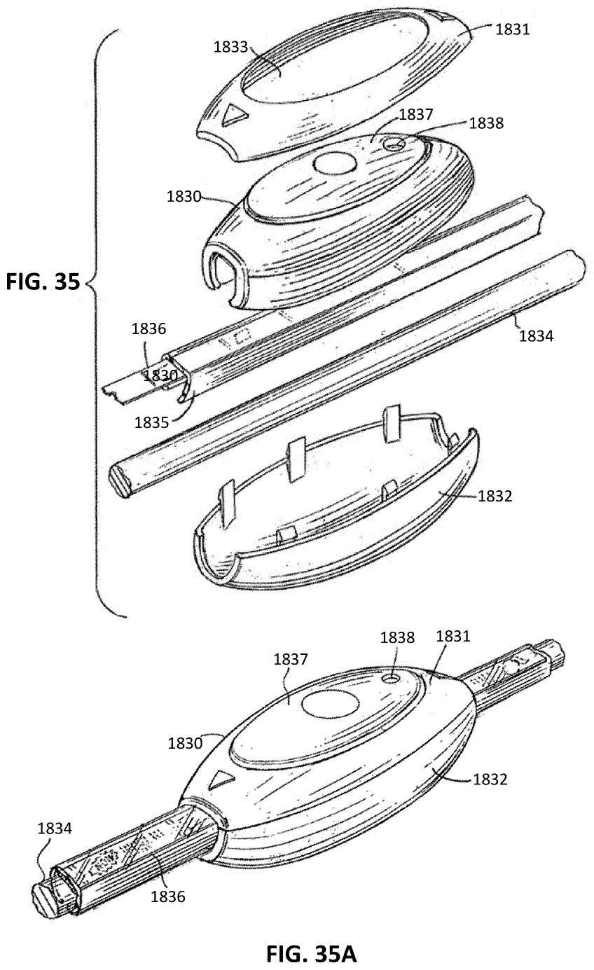

In one embodiment, illustrated in FIGS. 34 and 35 the sanitizing device is not mounted directly on the handle bar 1834, but rather is mounted on a sheath 1835 which is, in turn, mounted on the handle bar 1834. In one embodiment, the sheath 1835 has a rectangular upper surface comprising a clear (transparent) material such as plastic. By detachment of the various components, the strip 1836 can be replaced when desired. As a non-limiting example, the sanitizing device can be made to move along the handle bar 1834, such that the entire length of the surface of the sheath 1835 can be cleaned or sanitized.

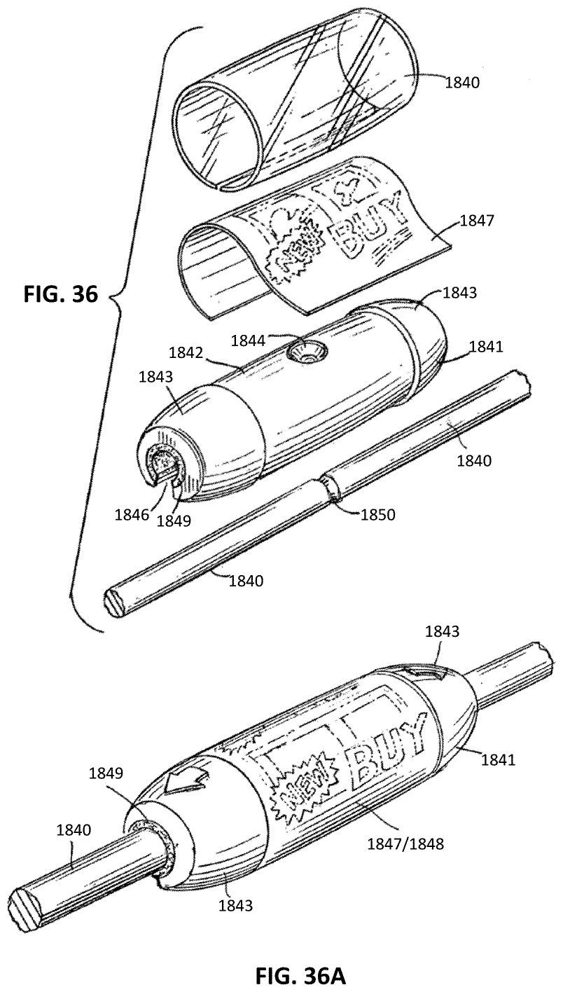

In one embodiment an applicator device 1841 for dispensing sanitizing layer/coating, illustrated in FIG. 38 includes a central section 1842 and end sections 1843 positioned on either side of the central section 1842. A fill port 1844 is provided. Fill port 1844 can be in an upper surface at the central section 1842, so that a reservoir, housed within the applicator device 1841 can be re-filled. A valve, cap or stopper can be included in the fill port 1844 to prevent leakage of sanitizing layer/coating from the reservoir. In one embodiment, a slot or passage 1846 runs along a length of the applicator device 1841 on a lower surface of the device opposite to the fill port 1844. The applicator device 1841 may be positioned with the slot or passage 1846 over, as a non-limiting example, a bar such as a handle bar 1840, and can then be snap-fitted around the bar, as is illustrated in FIG. 36.

The sanitizing layer/coating can be a liquid, a powder form, a combination thereof and the like.

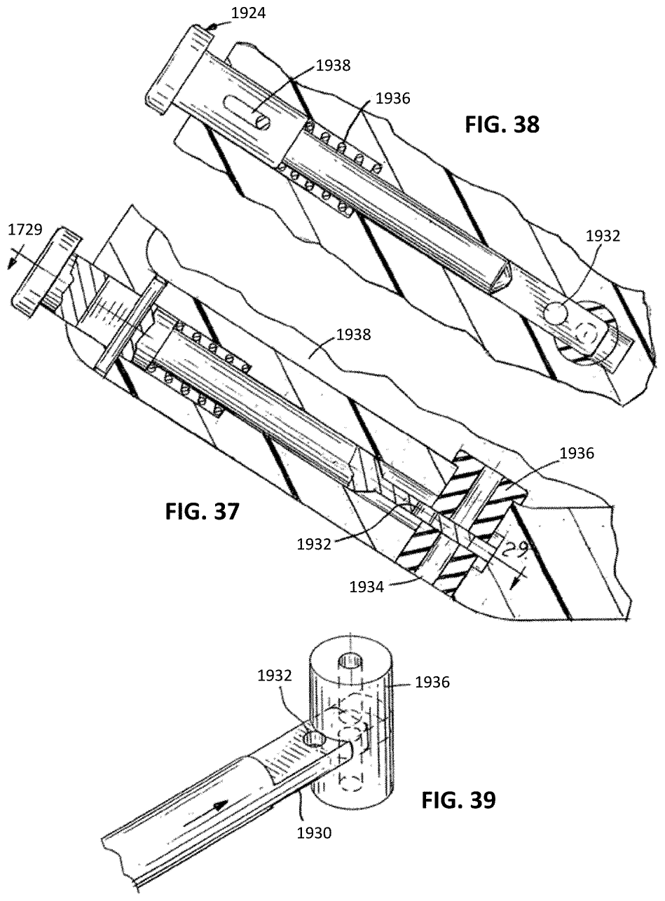

FIGS. 37 through 39 illustrate another embodiment of a valve. A plunger device 1924 is used. As a non-limiting example, the plunger device 1924 is biased outwardly by a spring 1926. The plunger device 1924 can be keyed or non-rotatably located to the passage 1928 and the distal end 1930 of the plunger device 1924 can include a through bore 1932 which may be aligned with a passage 1934 in the tubular member 1936 leading from reservoir 1938. Depressing the plunger device 1924 enables sanitizing layer/coating to flow from the reservoir 1938 by gravity flow through the passage 1934.

In another embodiment, illustrated in FIG. 39, a supplemental discharge mechanism enables the user of the applicator device 1841 to sanitize their hands, by way of example, or by way of example to place sanitizing layer/coating on a tissue and effectively wash their hands using the sanitizing agent.

In one embodiment, the applicator device 1841 may include one or more UV light sources.

It is to be understood that the present disclosure is not to be limited to the specific examples illustrated and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims. Accordingly, parenthetical reference numerals in the appended claims are presented for illustrative purposes only and are not intended to limit the scope of the claimed subject matter to the specific examples provided in the present disclosure.

What is claimed is:

1. An electric vehicle, comprising:
a steering element that can include a handle bar element;
a platform configured to support a rider of the electric vehicle;
at least one front Wheel and at least one rear wheel;
an electric motor configured to provide a mechanical power to at least one of the front and rear wheels;
a battery housing configured to removably receive and hold one or more battery packs, wherein the one or more battery packs are configured to power the electric vehicle; and
an applicator device for dispensing sanitizing layer/coating to at least a portion of the steering element.

2. The vehicle of claim 1, wherein the applicator device includes a central section and end sections positioned on either side of the central section.

3. The vehicle of claim 2, further comprising:
a fill port coupled to the applicator device.

4. The device of claim 3, wherein the fill port is positioned at an upper surface at the central section.

5. The device of claim 4, wherein the fill port is coupled to a sanitizing layer/coating reservoir.

6. The device of claim 1, wherein the sanitizing layer/coating reservoir is positioned in a housing of the applicator device.

7. The device of claim 6, wherein the sanitizing layer/coating reservoir is configured to be re-filled.

8. The device of claim 7, wherein the fill port includes at least one of: a valve; cap and stopper.

9. The device of claim 8, wherein the at least one of: a valve; cap and stopper is included in the fill port to prevent leakage of sanitizing layer/coating from the reservoir.

10. The device of claim 9, further including:
a passage that runs along a length of the applicator device.

11. The device of claim 10, wherein the passage runs on a lower surface of the applicator device.

12. The device of claim 11, wherein the passage runs on a lower surface of the applicator device opposite to the fill port.

13. The device of claim 12, wherein the applicator device is positioned with the slot or passage over the handle bar.

14. The device of claim 12, wherein the applicator device is snap-fitted around the handle bar.

15. The device of claim 1, wherein the sanitizing layer/coating is selected from one or more of: a liquid; a powder; and a combination thereof.

16. The device of claim 1, further comprising:
a plunger device coupled to the applicator device.

17. The device of claim 16, wherein the plunger device is biased outwardly.

18. The device of claim 16, wherein the plunger device is biased outwardly. by a spring.

19. The device of claim 16, wherein the plunger device is non-rotatably located relative to a passage.

20. The device of claim 16, wherein depressing the plunger device enables sanitizing layer/coating to flow.
